(19) United States
(12) Patent Application Publication
(10) Pub. No.: US 2023/0028948 A1
(43) Pub. Date: Jan. 26, 2023

(54) GENERATION OF PATIENT-SPECIFIC CONTENT

(71) Applicant: GIST MED LTD, Tel Aviv (IL)

(72) Inventors: Dan ROLLS, Tel Aviv (IL); Amir BEKER, Rosh Haayin (IL); Orit NEUDORFER, Tel Aviv (IL)

(21) Appl. No.: 17/790,568

(22) PCT Filed: Dec. 31, 2020
(86) PCT No.: PCT/IL2020/051368

§ 371 Date: Jul. 1, 2022

(30) Foreign Application Priority Data
Jan. 2, 2020 (IL) 271815

(51) Int. Cl. G16H 10/60; G16H 30/20; G16H 50/70

(52) U.S. Cl. CPC G16H 10/60; G16H 30/20; G16H 50/70

(57) ABSTRACT

The present disclosure discloses a method and system for generating a patient-specific content to a patient undergoing medical procedure. The system and the method of the present disclosure receives data related to the patient from various input sources, e.g. care-givers, the patient or data of medical attributes of the patient fed directly by monitoring or diagnostic devices, etc. The system stores a plurality of prior-prepared and existing media files, e.g. video or audio files, each of which may be related to a specific patient health parameter, condition or intended medical procedure or treatment that patient is or will undergo. Based on the specific input data of a patient, the system generates a patient-specific media content, or also can be referred to as personalized content objects (PCOs), that is constituted from at least two of said media files. The patient-specific media content includes a sequence of media files that typically has a specific chronological and/or narratively order commensurate with the flow of the intended medical procedure or treatment; the patient is exposed to the content before and/or during and/or after the medical treatment or procedure. This content may be displayed to the patients in sequences, before or during each phase, or the entire content may be displayed as one continuum at the choosing of the patients. The content may be displayed on dedicated screens accessible by or visible to the patients or may be delivered to him through a patient-carried device, e.g. a smart phone or tablet computer.

### TECHNOLOGICAL FIELD

The present disclosure is in the field of personalized systems for medical information accessibility to a patient.

### BACKGROUND

To make appropriate health decisions and act on them, individuals need to identify health information, understand the content, and evaluate the information for credibility and quality. Many factors affecting the ability to process and understand health information by individuals and populations. These factors include age, gender, educational level, literacy rate, illnesses, socioeconomic status, occupation, knowledge of the language, cultural background, presence of physical disabilities or cognitive impairment. Patients undergoing medical procedures are loaded with information, before, during and after the medical procedure is carried out. In many cases, the patient cannot absorb all the information and is left with a lot of open questions and at times it may also affect on the recovery process following the procedure. Some patients overcome it with information they are collecting from the web, which may be confusing and at times even misleading. Proper accessibility to medical-related information may improve the experience of the patient and assist to go through the process.

### GENERAL DESCRIPTION

The present disclosure discloses a method and system for generating a patient-specific content to a patient undergoing medical procedure. The system of the present disclosure receives data related to the patient from various input sources, e.g. care-givers, the patient or data of medical attributes of the patient fed directly by monitoring or diagnostic devices, etc. The system stores a plurality of prior-prepared and existing media files, e.g. video or audio files, each of which may be related to a specific patient health parameter, condition or intended medical procedure or treatment that patient is or will undergo. Based on the specific input data of a patient, the system generates a patient-specific media content, or also can be referred to as personalized content objects (PCOs), that is constituted from at least two of said media files. The patient-specific media content includes a sequence of media files that typically has a specific chronological and/or narratively order commensurate with the flow of the intended medical procedure or treatment; the patient is exposed to the content before and/or during and/or after the medical treatment or procedure. For example, for a patient undergoing a medical intervention procedure involving a preparatory phase, intervention phase and a recovery phase, the patient may receive content explaining the details of the preparatory phase, what the intervention procedure is and what exactly is being done and how fast the recovery is, what side effects to expect, etc. In another example, a patient underwent a medical procedure may interacts with the system at his/her home to receive a specific content that includes explanations of the recovery process, reminders of measurements the patient need to perform and reports that need to be sent to the caregiver, etc. It is to be noted that the system may be configured to receive inputs from the patient, e.g. measurements of physiological parameters, and store it. This content may be displayed to the patients in sequences, one displayed before or during each phase, or the entire content may be displayed as one continuum at the choosing of the patients. The content may be displayed on dedicated screens accessible by or visible to the patients or may be delivered to him through a patient-carried device, e.g. a smart phone or tablet computer.

The method and the system of the present disclosure aim to improve the patient engagement during any phase of a medical-related procedure.

An aspect of the present disclosure provides a method for generating a patient-specific content for guiding a patient through a medical intervention procedure. The method includes receiving at least one patient-related parameter either by automatic techniques or by manual input. The input may be received in various ways, such as (i) an input of the patient or a caregiver through a designated user interface; (ii) an audio input, e.g. by recording the explanations of a physician to a patient and translating it to a patient-related parameter; (iii) an input of a medical examination data, either in a written report format, images or any other raw data. The method further includes selecting from a database of media file building blocks at least two media files, of which the metadata thereof is associated with the received patient-related parameters. Each media file in the database is indexed with one or more specific patient- or intervention-related metadata. Upon satisfying a correlation condition between a parameter of the patient and a media file, the media file is being selected and hence forms a part in the patient-specific media content.

The selected media files are combined into a patient-specific media file, to thereby generate a patient specific content. The patient-specific media file is transmitted to be outputted to the patient. Optionally, the method further includes outputting the patient-specific media file to the patient. The patient-specific media file (interchangeably used herein with the term “patient-specific content”) is a content that is tailored to the patient according to the input of the patient-related parameters and is typically formed of a sequence of videos that provide the patient essential information regarding the medical procedure, the consequences thereof or any other related information that may be of use to the patient. The content may replace human resources of professional team that is typically in charge of providing this information to the patient.

Typically, the patient-specific content is formed of a sequence of a plurality of media file building blocks combined at a certain sequence to provide the patient all the essential data he/she requires to understand the expected medical procedure. The media file building blocks are selected according to the patient-related parameters being inputted. The characteristics of the patient-related parameters trigger the selection of specific media files building blocks out of the existing database and the chronological sequence they appear. Thus, each patient is expected to be presented with a tailor-made patient-specific content.

In some embodiments of the method and/or the system as described below, the at least one patient-related parameter is one of procedure type (e.g. the type of the surgery), patient identification details, gender, age, contact information or any combination thereof.

In some embodiments of the method and/or the system as described below, the selected media files building blocks comprise interactive content that requires the patient's input, e.g. replying on a questionnaire. The method further includes receiving further input from the patient in response to the interactive content and optionally generating an updated patient-specific content based on the inputs of the patient (e.g. the answers to the questionnaire), invoking a new or modified selection of media files building blocks. The interactive content may include signing on various medical-legal documents authorizing the performance of the medical procedure, such as an informed consent document.

In some embodiments, the method includes, following an output of the specific media file to the patient, inputting additional patient-related parameter or updating existing patient-related parameter that is previously received to generate and output an updated patient-specific content to the patient. This may be a result of new data that is obtained during the medical procedure the patient undergoes. In other words, at any stage of the medical procedure, the patient-specific content may be updated according to updated medical data related to the patient that are determined according to clinical decisions being made. The patient can view at any point all content of the specific media file, whether it is related to the history of the medical procedure or to a medical procedure to be followed in the future.

In some embodiments of the method, the patient-related parameters are selected from any one of the following: age, gender, education, socio-economics, ethnicity, geographical location, language, medical condition, physiological symptoms, medical procedure, current and planned medical treatment, medical history, hospitalization history, health literacy level, patient cultural and social attributes, physiological or behavioral parameters and any combination thereof. Each of the parameters may affect the eventual patient-specific media content. For example, based on the geographical location of the patient, the patient-specific media content is adapted to conform with a local style of speaking, etc. In some embodiments of the method, the media files are audio files, video files and/or static media files, such as images, graphic information sheets, documents, etc. The video files may be animations that includes characters that provides the data to the patient

In some embodiments of the method, the media files are static media files, such as images, graphic information sheets, documents, etc.

In some embodiments, the method further comprising matching each of the selected media file building block with an audio file from an audio database. Each audio file in the database comprises one or more first matching criteria and each media file building block comprises corresponding one or more second matching criteria. The matching is carried out based on a fitting model between the first and the second matching criteria. In other words, the method includes matching audio content to each media file building block that is selected according to the required atmosphere or any other specific attribute of the content that is being presented. The matching can be performed for several consecutive selected media file building blocks or to a single one.

In some embodiments of the method, at least some of the media file building blocks include adaptive sections, namely specific regions on a video file or adaptive content screens that are adapted specifically for each patient according to the specific parameters of the patient that are inputted. For example, a media file may include instructions for taking medications or for utilizing medical devices. The instructions may include dosage regiment that are being adapted for each patient based on its age, weight, medical conditions, etc., and/or the explanations about the medical device and the specific recommended practice of its use for the patient. A patient-specific adaptive section may be selected from prior-prepared options or may be selected based on an algorithm, e.g. calculation of dosage regiment, or determined by a specific input. Furthermore, in some embodiments, the adaptive sections may include language selection, namely the language is chosen according to the patient, reference to the gender of the patient, its culture, ethnics, etc.

In some embodiments of the method or the system as described below, the patient-related parameter comprises the medical condition or pathology of the patient and the media files building blocks are selected to output a patient specific content specifically that provides explanation of the patient's pathology or medical condition to the subject. The patient specific content may include one or more media files providing background explanations of the organ and/or visual and/or written informative data of the anatomical system and/or the physiological process or pathway related to the pathology/medical condition. For example, the background may include explanations of the role of the organ or system in the human body and where it is located. Furthermore, the selected media files building blocks may include illustrations and/or explanations of the specific improper functionality of the organ/anatomical system and the proposed procedure the patient is expected to undergo to fix it. Thus, the patient specific content may be tailored specifically to facilitate the patient understanding his/her medical condition and the expected procedure.

In some embodiments of the method and/or the system as described below, at least one of the patient-related parameters is a medical input from a medical examination that may be received either directly from the medical examination tool, e.g. in a raw data format, or in the form of a medical report, such as a report of a medical imaging or other diagnostic or monitoring procedure of the patient that includes medical analysis of the identified pathologies demonstrated by the imaging or the other procedure. For example, the patient may undergo a CT or Ultrasound imaging and the findings of the imaging is detailed in a written report by a professional. The report may be received as a patient-related parameter and it is analyzed, e.g. by a Natural Language Processing (NLP) to extract the relevant medical data therefrom. The media file building blocks are selected based on the information that is extracted from the medical report to create an illustration of the pathology for the patient. The

Thus, in some embodiments, the method is further comprising and/or the processing circuitry of the system as described below is further configured for receiving a processed information of a medical report of an imaging or other diagnostic or monitoring procedure of the patient and selecting media file building blocks for providing a patient-specific content that comprises visual illustration related to said imaging or procedure. The patient-specific content may include an explanation of the imaged organ or anatomical system, illustration of the identified pathology and/or explanation of the treatment to be followed. The illustration may be 2-D or 3-D illustration of the imaged organ/anatomical system/physiological process or the identified pathology.

In some embodiments, the method comprising and/or the processing circuitry of the system as described below is further configured receiving a medical report and extracting said at least one patient-related parameter from said medical report. The extraction may be performed by any known technique such as NLP or machine learning techniques.

In some embodiments, at least some of the media file building blocks include a changeable non-informative data. The changeable non-informative data may be a decorative background to the clinical information that is presented in the media file or any element that effect the atmosphere, feeling of the media file. The changeable non-informative data may change randomly between patients or upon certain parameters, such as time of the day, day in the week or based on some patient-specific parameters.

In some embodiments, the method further includes generating a patient-specific engagement profile. Namely, based on the patient-related parameters, content is presented to the patient, for example, at a certain frequency, certain days in the week, certain time in the day, at certain repetition pattern of specific media content, etc.

In some embodiments, the specific patient- or intervention-related metadata are selected from any one of the following: nature of drug treatment, type of medical procedure, medical instruction relating to lifestyle or nutrition, potential outcome of the medical intervention, safety information and any combination thereof.

In some embodiments of the method and/or the system as described below, the media files building blocks comprise content instructions or recommendations for administration of pharmaceuticals and/or additives during the medical procedure.

In some embodiments of the method and/or the system as described below the media files building blocks comprise content of instructions or recommendations for the patient for utilizing a medical device during the medical procedure.

In some embodiments of the method and/or the system as described below, the patient-related parameter comprises pharmaceuticals or additives that are consumed regularly by the patient independent to the procedure he/she are going through. This can trigger a selection of media files building blocks that include information of pharmaceuticals or additives that should be avoided in view of new pharmaceuticals that are to be consumed by the patient during the medical procedure.

In some embodiments of the method and/or the system as described below, the patient-related parameter comprises new pharmaceuticals or additives to be consumed by the patient during the medical procedure. In some embodiments, the method further includes verifying the type and dosage of said pharmaceutical and/or additive and if an anomaly or a mistake is identified than the method further includes selecting media files building blocks that comprises one or more alerts for the patient indicative of said identified anomaly or mistake.

It is to be noted that the method may include several iterations during the medical procedure that a patient-related parameter is being received. For example, it can be received at the beginning of the procedure and the personal content that is generated for the patient related to the expected procedure for the patient. Additional patient-related parameter may be received at the end of the medical procedure, and the personal content is updated based thereon.

In some embodiments of the method, the combined media file is selectable or accessible by a patient-specific authorization access key, e.g. a code, a bar-code, a QR code, a typed code, fingerprint, facial features, etc. Namely, only authorized individuals are permitted to access the patient-specific media file to maintain the medical-related data classified.

In some embodiments, the combined media file is distributable to different output devices. The combined media file is selectable at each device by the patient-specific authorization access key.

In some embodiments, the method includes determining matching correlation of the at least one patient-related parameter of the patient with predetermined patient-related parameter data of other patients and wherein said selecting and/or combining is at least partially based on the degree of correlation. In other words, a profile of the patient is generated based on all the received inputs and the method includes determining a degree of correlation with other patients' profiles that are predetermined. High degree of correlation may affect the patient-specific content based on the data related to the matching patient, e.g. the engagement profile of the patient. For example, patients with similar certain ethnic origins may be assumed to have similar engagement profiles.

Another aspect of the present disclosure provides a content management system for generating and outputting patient-specific content for guiding a patient through a medical intervention procedure. The system includes a parameter module for receiving at least one patient-related parameter. The system further includes a database of computer-readable media file building blocks. Each computer-readable media file building blocks is indexed with one or more specific user- or intervention-related metadata. A processing circuitry of the system is configured for (i) selecting at least two media files, the metadata of which is associated with said patient-related parameters and (ii) combining the at least two media files into a combined, patient-specific media file to thereby generate a patient specific content. The processing circuitry is configured to select media files from the database upon identifying satisfaction of predetermined correlation conditions between the patient-related parameter and the metadata of the specific media file building block.

In some embodiments, the system further includes an output unit for outputting the combined and patient-specific media file to the patient.

In some embodiments, the processing circuitry is further configured for generating a patient-specific engagement profile. Namely, based on the patient-related parameters or on other parameters related to patient engagement, content is presented to the patient, for example, at a certain frequency, certain days in the week, certain time in the day, at certain repetition pattern of specific media content, etc. It is noted that there may be a default engagement profile that is pre-defined in the system.

In some embodiments of the content management system, the output unit includes one or both of an audio or visual output device, e.g. loudspeaker, display.

In some embodiments, the system includes a data-retrieval module for retrieving patient-related parameters out of the patient-related medical records, i.e. from a database of a medical center that has medical data of the patient. The patient-related parameters are being fed to the parameter module and the processing circuitry is configured to select and combine the media file building blocks based on the patient-related parameters retrieved from medical records in combination with other parameters received in the parameter module.

In some embodiments of the system, the parameter module includes one or more input interfaces, e.g. interactive interfaces that requires input by individuals, for inputting the patient-related parameters.

In some embodiments of the content management system, the one more input interfaces include a patient interface and a caregiver interface.

In some embodiments of the content management system, the patient interface is configured for inputting patient physiological parameters, behavioral parameters and medical information. The parameters may be collected passively, e.g. via sensors such as a microphone, or actively e.g. by a response inserted directly to the system by the patient, e.g. measurements of physiological parameters of the patient that are taken at home.

In some embodiments of the system, the processing circuitry is configured for analyzing parameters relating to patient engagement, learning, compliance, request or feedback from the patient. Based on the analysis of the parameters relating to patient engagement learning, compliance, request or feedback from the patient, the processing circuitry may be configured for adapting the patient engagement-profile to increase the possibility of comprehension of the content that is delivered to the patient.

The parameters relating to patient engagement, learning, compliance, request or feedback from the patient may be collected either automatically, i.e. by sensors, engagement activities of the patient, etc., or manually by manual input of the patient or caregiver, e.g. by a Q&A session.

In some embodiments of the system, the processing circuitry is further configured for matching each of the selected media file building block with an audio file from an audio database. Each audio file in the database comprises one or more first matching criteria and each media file building block comprises corresponding one or more second matching criteria. The matching is carried out based on a fitting model between the first and the second matching criteria. In other words, the processing circuitry is configured for matching audio content to each media file building block that is selected according to the required atmosphere or any other attribute of the content that is being presented. The matching can be performed for several consecutive selected media file building blocks or to a single one.

In some embodiments of the system, the processing circuitry is configured for calculating a patient engagement score/index based on the analyzed parameters. The score is based on initial parameters and it is dynamic over time. The score is calculated based on the patient's data, as well as relative to other comparable patients. The score may relate to a single patient or a population of patients. The processing circuitry is configured for adapting the patient-specific media file or the engagement profile based on the calculated engagement score. For example, upon change of the engagement score of the patient, the frequency of content presented or sent to the patient varies. In some embodiments, the engagement score of the patient may affect the playback speed of the content being presented to the patient.

In some embodiments of the system, the one or more interfaces are configured for receiving a patient-specific authorization access key, e.g. a patient-specific code, a bar-code, a QR code, a typed code, fingerprint, facial features, etc. The combined media file is selectable by the patient-specific authorization access key code.

In some embodiments, the system includes a plurality of output devices. The output devices may be distributed at different locations that may be encountered by the patient or accompanying persons during the intervention procedure, including for example, at home.

In some embodiments of the system, the media files are video files, audio files and/or static media files, such as images, graphic information sheets, documents, etc. The video files may be animations that includes characters that provides the data to the patient.

In some embodiments of the system, at least some of the media file building blocks include adaptive sections that are adapted according to the specific parameters of the patient. For example, a media file may include instructions for taking medications. The instructions may include dosage regiment that are being adapted for each patient based on its age, weight, medical conditions, etc. A patient-specific adaptive section may be selected from a prior-prepared options or may be selected based on an algorithm, e.g. calculation of dosage regiment, or determined by a specific input.

In some embodiments, at least some of the media file building blocks include a changeable non-informative data. The changeable non-informative data may be a decorative background to the clinical information that is presented in the media file or any element that effect the atmosphere, feeling of the media file. The changeable non-informative data may change randomly between patients or upon certain parameters, such as time of the day, day in the week or based on some patient-specific parameters.

In some embodiments of the system, the processor includes a patient-matching module configured for (i) determining matching correlation of the at least one patient-related parameter of the patient with predetermined patient-related parameter data of other patients (ii) affecting the selection and/or combination of the processor based on the degree of correlation. The patient-matching module is configured for identifying a correlation between the at least one patient-related parameter of the patient and those of other patients that are prestored in a database. Upon finding a matching correlation of a certain degree between the patient of interest and other patients, the eventual patient specific content is affected by engagement data related to the correlated patient in order to increase the patient engagement.

Yet another aspect of the present disclosure provides a computerized assembly, for generating and outputting patient-specific content for guiding a patients through a medical intervention procedure. The computerized assembly includes (a) a parameter module for receiving at least one patient-related parameter; (b) a communication module for communicating with a database of computer-readable media file building blocks, each of which being indexed with one or more specific user- or intervention-related metadata; and (c) a processor configured for (i) selecting at least two media files, the metadata of which is associated with said user-related parameters, (ii) actuating said communication module to retrieve said at least two media files, and for (iii) combining the at least two media files into a combined, patient-specific media file to thereby generate a patient specific content.

In some embodiments, the computerized assembly includes an output unit for outputting the combined and patient-specific media file to the patient.

In some embodiments, the processing circuitry is further configured for generating a patient-specific engagement profile. Namely, based on the patient-related parameters, content is presented to the patient, for example, at a certain frequency, certain days in the week, certain time in the day, at certain repetition pattern of specific media content, etc.

In some embodiments of the computerized assembly, the output unit includes one or both of an audio or visual output device, e.g. a loudspeaker or a display including a mobile device, tablet, computing unit, etc.

In some embodiments, the computerized assembly includes a data-retrieval module for retrieving patient related parameters out of the patient-related medical records and for feeding it to the parameter module.

In some embodiments, the computerized assembly includes a code module for inputting an authorization access key, e.g. a patient-specific code, and wherein the combined media file is selectable by the authorization access key.

In some embodiments, the computerized assembly includes a plurality of output devices distributed at different locations that may be encountered by the patient or accompanying individuals during the intervention procedure, including, for example, at home.

In some embodiments of the computerized assembly one or more of the output devices is associated with a code module for inputting the patient-specific authorization access key to thereby permit the output device to output said patient-specific media file.

In some embodiments, the computerized assembly is configured for use in medical centers. In some embodiments, the computerized assembly is configured for use in increasing patient engagement.

Yet another aspect of the present disclosure provides a patient-specific media file that is constituted by at least two selected media file building blocks each of which being indexed with one or more specific user- or intervention-related metadata, the metadata of which is associated with predefined user-related parameters.

In some embodiments, the patient-specific media file is generated by selecting from a database of media file building blocks, each of which being indexed with one or more specific patient- or intervention-related metadata, at least two media files, the metadata of which is associated with parameters of the patients; and combining the at least two media files into a combined, patient-specific media file to thereby generate a patient specific content.

An aspect of the present disclosure provides a plurality of the above-described patient specific media, wherein the media files are all attributed to the same patient. In some embodiments of the plurality of patient specific medial files, the combined media file is selectable by a patient-specific code.

In some embodiments of the plurality of patient specific medial files, the media files are all attributed different patients with at least 50%, 60%, 70%, 80%, 90%, 95% or 98% similarity in their user- or intervention-related metadata.

In some embodiments of the plurality of patient specific medial files, the media file building blocks are further indexed with metadata related to a feedback from the patient, feedback from a caregiver, or based on the media files history of said patient.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a flow diagram of a non-limiting example of an embodiment of the method of the present disclosure.

FIG. 2 is a schematic illustration of a non-limiting and non-exhausting example of building blocks of associated content sorted into different medical-related verticals.

FIGS. 3A-3B are block diagrams of non-limiting examples of embodiments of the system of the present disclosure.

### DETAILED DESCRIPTION OF EMBODIMENTS

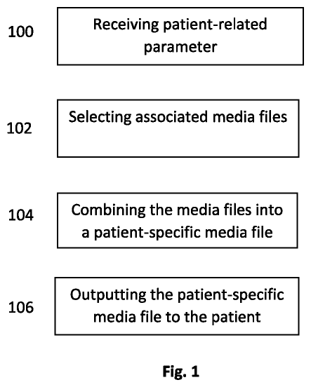

Reference is first made to FIG. 1, which is a flow-diagram exemplifying an embodiment of the method of the present disclosure. The method includes receiving one or more patient-related parameters 150. The patient-related parameter may include the type of treatment that the patient received, the diagnosed reason for hospitalization/the cause of disease, the treatment the patient received, instructions for release, etc. The parameters associated with the patient are each associated with media files that are selected 152 for the specific patient and combined 154 into a patient-specific media file. The patient-specific media file may include a chronologically-sequenced media files that are intended to be viewed by the patient in the specific sequence. The method further includes outputting the patient-specific media file 156 to the patient, e.g. by an audio and/or video based output device. It is to be noted, that each media file constituting a part of the patient-specific media file may be accessible by the patient at any time, or at some instances, some of the media files may be accessible merely at specific and predetermined time frames.

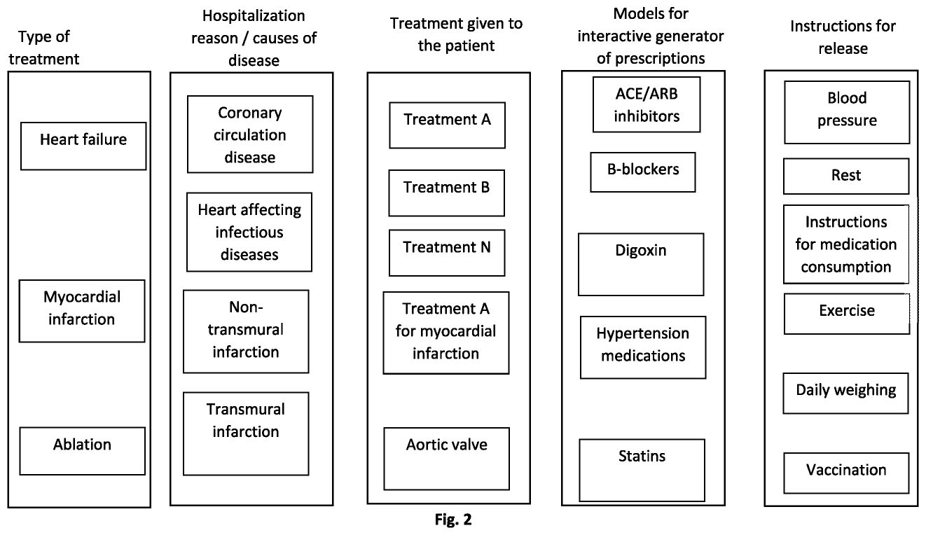

FIG. 2 shows an example of patient-related parameters that are sorted in different verticals. Each patient-related parameter is associated with one or more specific media file contents. The selected media file content may have many forms, for example, it can be adapted to the language of the patient, its culture, age, gender, ethnic or being adapted according to local standards (e.g. a manner a procedure is performed, release protocols), etc. The patient can be characterized by one or more parameters from each vertical, based on care givers input or measured parameters from different measuring devices measuring the patient, e.g. blood pressure, heart rate, etc.

The patient-specific media file may be formed by a plurality of media files that are associated with the parameters related thereto, each media file is associated with one or more parameters from each vertical. The patient receives specific content that intended to him/her that can be accessed through an output device. The patient-specific media file grants the patient guidance and information for the medical procedure he/she is going through. At each stage of the procedure, e.g. prior to a surgery or following the release from the hospital, the patient-specific media file may suggest a different media file to be viewed by the patient according to his/hers present medical condition/state.

It is to be noted that FIG. 2 shows merely an example of parameters that are associated with media file building blocks. Practically, there may be much more options for patient-related parameters that may be arranged under different verticals. Furthermore, it is to be noted that the sources of the input of the patient-related parameters may change between different patients and different procedures.

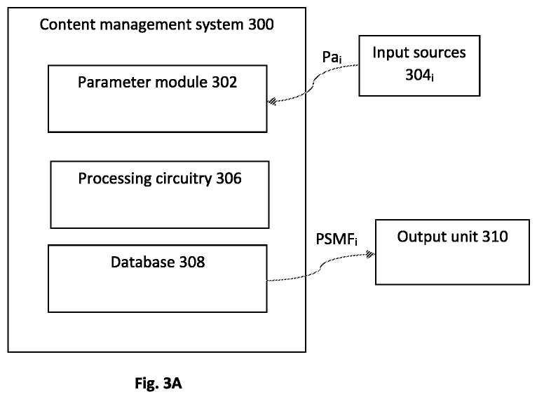

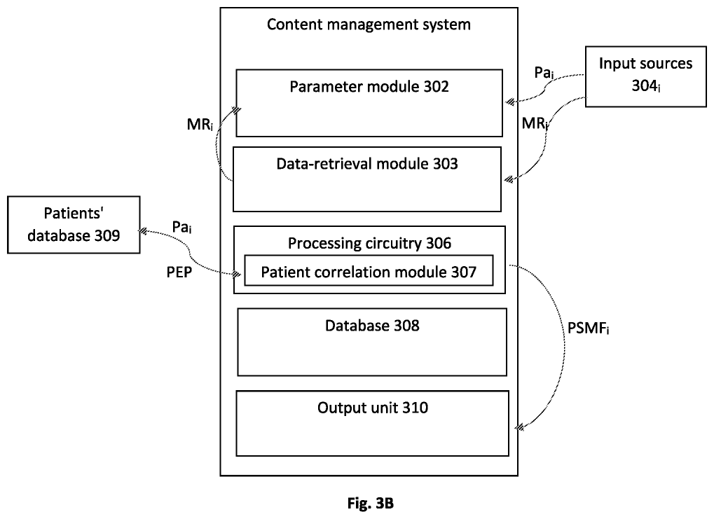

FIGS. 3A-3B are block diagrams of exemplary embodiments of the system of the present disclosure. FIG. 3A exemplify a content management system 300 for generating patient-specific content for each patient. The system 300 is configured to generate a patient-specific media file for each patient that is customized based on specific input parameters that are specifically-related to the patient. The content management system 300 includes a parameter module 302 configured for receiving one or more parameters Paᵢ related to a patient. The parameters Paᵢ are received from various input sources 304ᵢ such as medical care centers databases, medical devices that monitor the patient, manual input of care givers, etc.

A processing circuitry 306 of the system 300 is configured to analyze the parameters related to the patient and select specific media files from media files database 308 that includes a plurality of prepared and ready-to-use media files. The processing circuitry 308 combines the selected media files to a patient-specific media file content that includes relevant medical data for the patient, e.g. explanation about the procedures he/she are about to or already gone through, guidance for administering medications or instructions following the release from the medical care center, etc. The processing circuitry may be configured to combine the media files at a certain logic order to be viewed by the patient at different time frames of the procedure. In some embodiments, the patient may access all the relevant content at any time and in some other embodiments of the system each media file is accessible only at certain, predetermined, time frames of the medical procedure. It is to be noted that the patient-specific media file may be updated upon any update or a change of any parameter of the patient. The patient-specific media file PSMFᵢ is transmitted to an output unit 310 configured to output the device to the patient. The outputted patient-specific media file PSMFᵢ includes video and audio data and the output unit 310, e.g. a monitor, a smartphone or a tablet, is configured to support the complete presentation of the patient-specific media file PSMFᵢ.

FIG. 3B is another example of an embodiment of the system of the present disclosure. This example differs from that described with respect to FIG. 3A by that the system includes the output unit 310 and further includes a data-retrieval module 303. FIG. 3B details that the input of the parameters to the system is received via two different channels. The parameter module 302 receives parameters from input of individuals Paᵢ, e.g. care giver or the patient and the data retrieval module 303 is configured to receive data of medical records of the patient MRᵢ, e.g. from datacenter of the medical care center in which the patient is being treated. The medical records MRᵢ are transmitted to the parameter module 302 and the processing circuitry 306 generates the patient-specific media file based on the parameters received in the data-retrieval module 303 and the parameter module 302. The patient-specific media file PSMFᵢ generated by the processing circuitry 306 is transmitted to the output unit 310 for being outputted to the patient. Furthermore, in this exemplary embodiment the processing circuitry 306 includes a patient correlation module 307 that is configured to identify a correlation between the parameters Paᵢ of the patient and similar parameters of patients that are stored in patient's database 309. Upon identifying a correlation with data of patients in the database, the patient correlation module extracts the patient engagement profile PEP of the relevant patient from the database 309 and applies it, at least partially, to generate the patient-specific media file PSMFᵢ.

1.-54. (canceled)

55. A method for generating a patient-specific content for guiding a patient through a medical procedure, comprising:
receiving at least one patient-related parameter;
selecting from a database of prior-prepared media file building blocks, each of which being indexed with one or more specific patient- or intervention-related metadata, at least two media files, the metadata of which is associated with said patient-related parameters, wherein said media file building blocks are video files;
combining the at least two media files into a combined, patient-specific media file, to thereby generate a sequence of media files forming a patient specific video, said at least two media files having a specific chronological order commensurate with the flow of the intended medical procedure and are intended to be viewed by the patient in said order; and
outputting said combined and patient-specific media file to the patient.

56. The method according to claim 55, wherein said patient-related parameters are selected from age, gender, education, socioeconomics, ethnicity, language, medical condition, physiological symptoms, medical procedure, current and planned medical treatment, medical history, hospitalization history, health literacy level, patient cultural and social attributes, physiological or behavioral parameters and any combination thereof.

57. The method according to claim 55, wherein said intervention-related metadata are selected from nature of drug treatment, type of medical procedure, medical instruction relating to lifestyle or nutrition, potential outcome of the medical intervention, safety information and any combination thereof.

58. The method according to claim 55, wherein the combined media file is selectable by a patient-specific code;
wherein said combined media file is distributable onto different output devices and selectable at each device by said code.

59. The method according to claim 55, comprising generating a specific-engagement profile of the subject based on patient engagement parameters and/or the at least one patient-related parameter.

60. The method according to claim 55, wherein at least some of the media file building blocks comprise an adaptive data content, said adaptive content is selected from: instructions for taking medications and language selection, the method further includes adapting said adaptive data content based on the at least one patient-related parameter.

61. The method according to claim 55, comprising determining matching correlation of the at least one patient-related parameter of the patient with predetermined patient-related parameter data of other patients and wherein said selecting and/or combining is at least partially based on the degree of correlation.

62. The method of claim 55, comprising matching each of the selected media file building block with an audio file from an audio database, each audio file in the database comprises one or more first matching criteria and each media file building block comprises corresponding one or more second matching criteria, said matching is carried out based on a fitting model between the first and the second matching criteria.

63. The method of claim 55, comprising receiving a medical examination data that comprises said at least one patient-related parameter;
wherein said medical examination data comprises an analysis of a medical report;
wherein said medical report is a medical imaging report.

64. A content management system for generating and outputting patient-specific content for guiding a patient through a medical intervention procedure, comprising:
a parameter module for receiving at least one patient-related parameter;
a database of computer-readable media file building blocks, each of which being indexed with one or more specific patient- or intervention-related metadata;
a processor configured for (i) selecting at least two media files, the metadata of which is associated with said patient-related parameters and (ii) combining the at least two media files into a combined, patient-specific media file to thereby generate a patient specific content; and
an output unit for outputting said combined and patient-specific media file to the patient.

65. The system of claim 64, comprising a data-retrieval module for retrieving patient related parameters out of the patient-related medical records and for feeding it to said parameter module;
wherein the parameter module comprises one or more input interfaces for inputting said patient-related parameters.

66. The system of claim 64, configured for analyzing parameters relating to patient engagement, learning, compliance, request or feedback from the patient; and
wherein the system is configured for calculating a patient engagement score/index based on said analyzed parameters;
wherein the processor is configured to generate a patient-specific engagement profile based on parameters relating to patient engagement, learning, compliance, request or feedback from the patient;
wherein the patient-specific engagement profile is configured to be updated based on a patient engagement score/index that is calculated based on said analyzed parameters.

67. The system of claim 64, wherein at least some of the media file building blocks comprise an adaptive data content, said adaptive content is selected from: instructions for taking medications and language selection, the processor is further configured for adapting said adaptive data content based on the at least one patient-related parameter.

68. The system of claim 64, wherein the processor comprises a patient-matching module configured for (i) determining matching correlation of the at least one patient-related parameter of the patient with predetermined patient-related parameter data of other patients (ii) affecting the selection and/or combination of the processor based on the degree of correlation.

69. The system of claim 64, wherein said parameter module is configured for receiving an analysis of a medical report that comprises said at least one patient-related parameter;
wherein said medical report is a medical imaging report
wherein said patient-specific media file comprises illustration and/or information related to identified pathology of the patient.
